(19) United States
(12) Patent Application Publication
(10) Pub. No.: US 2019/0234944 A1
(43) Pub. Date: Aug. 1, 2019

(54) DEVICES AND METHODS TO REDUCE INTERFERING COMPOUNDS IN BIOLOGICAL SAMPLES

(71) Applicant: LABORATORY CORPORATION OF AMERICA HOLDINGS, Burlington, NC (US)

(72) Inventor: Kelly Y. Chun, Burlington, NC (US)

(21) Appl. No.: 16/338,807

(22) PCT Filed: Oct. 13, 2017
(86) PCT No.: PCT/US17/56584

§ 371 Date: Apr. 2, 2019

Related U.S. Application Data
(60) Provisional application No. 62/407,785, filed on Oct. 13, 2016.

(51) Int. Cl. G01N 33/543

(52) U.S. Cl. CPC G01N 33/54393; G01N 33/54353; G01N 33/54306

(57) ABSTRACT

This disclosure provides devices and methods to reduce interfering compounds in biological samples. In one embodiment, disclosed is a device to reduce a target interfering compound in a biological sample comprising a binding agent attached to a solid support, the binding agent capable of binding a target interfering compound within the biological sample, wherein binding of the target interfering compound to the binding agent reduces the amount of target interfering compound in the biological sample. In other embodiments, provided is a method of reducing the amount of a target interfering compound in a biological sample comprising: adding a binding agent to the sample, wherein the binding agent binds the target interfering compound with a higher affinity than the binding agent binds to other components in the sample; incubating the sample to allow a complex to form between the binding agent and the target interfering compound; and removing the complexed target interfering compound that is bound to the binding agent.

### CROSS REFERENCE TO RELATED APPLICATION

This application claims the benefit of U.S. Provisional Application No. 62/407,785, filed Oct. 13, 2016, which is incorporated by reference in its entirety herein.

### FIELD OF THE INVENTION

This invention relates to devices and methods to reduce interfering compounds in biological samples.

### BACKGROUND

Biological samples may be analyzed to provide a wealth of information about the subject from which the sample was obtained. However, the accuracy of analytical tests are subject to interference by factors such as compounds present in the sample. Interfering compounds can skew or alter test results and thus create false positive or false negative results. Accuracy of analytical tests thus requires a means to control for or eliminate interfering compounds from biological samples.

Diet, medication, and other exposures introduce an enormous assortment of compounds in wide concentration ranges into a subject's body. Genetic dispositions may also contribute to the presence of undesirable or unnatural amounts of interfering compounds. However, the popularity of dietary supplementation has caused it to become a primary source of interfering compounds. Despite evidence of no benefit or even possible harm in some instances, supplementation of vitamins and minerals continues to grow. It is estimated that the US supplement industry is now worth over $30 billion annually. Although many supplements are conceptually designed to provide natural compounds and/or precursors or metabolites thereof, supplements often provide such compounds in unnatural, very high doses.

Technicians have always had to contend with a wide array of ingested materials with the potential to interfere with tests. Recently, however, biotin has become a pervasive and dominant interferent due in large part to biotin supplementation. Also known as vitamin B7 or vitamin H, biotin is an essential coenzyme involved in carbon dioxide transfer in carboxylase enzymes. The Recommended Daily Intake (RDI) of biotin is 30 μg per day, and supplementation is normally not necessary as biotin is ubiquitous in common foods. However, biotin has been successfully marketed as a beauty supplement in recent years. Since over-the-counter biotin-containing supplements and beauty products are unregulated, the amount of biotin a subject ingests can be extremely high. Supplements containing 3 mg or more of biotin suggest that consumers are ingesting amounts of biotin far in excess of the RDI. Even in large doses, biotin is considered nontoxic and is unlikely to cause any side effects. However, excess biotin can interfere with a broad range of analytical and diagnostic tests. Thus, there is a need to develop tools and methods to reduce interfering compounds, such as but not limited to biotin, in biological samples.

### SUMMARY

Embodiments disclosed comprise methods and devices which reduce levels of interfering compounds in biological samples, as well as methods of using such devices, and kits comprising such devices to reduce levels of interfering compounds in biological samples.

For example, disclosed is a device to reduce interfering compounds in a biological sample. The device may comprise a binding agent capable of binding a target interfering compound within the biological sample; wherein binding of the target interfering compound to the binding agent reduces the amount of target interfering compound in the biological sample, but does not significantly reduce the concentration of other analytes in the sample. In some embodiments, the binding agent is attached to a solid support. In some embodiments, the device may comprise a receptacle having inner walls that are attached to the binding agent. In an embodiment, the binding agent is attached to the solid support via a linker.

In some embodiments, the binding agent binds the target interfering compound with a dissociation constant (K_{d}) of about 10⁻¹⁰ M or less. In some embodiments, the binding agent comprises streptavidin, avidin, or an antibody. In some embodiments, the target comprises biotin and the biological sample comprises blood or plasma.

In one embodiment, the invention comprises a method of reducing interfering compounds in a biological sample. For example, disclosed is a method of reducing the amount of a target interfering compound in a biological sample comprising: adding a binding agent to the sample, wherein the binding agent binds the target interfering compound with a higher affinity than the binding agent bind to other components in the sample; incubating the sample to allow a complex to form between the binding agent and the target interfering compound; and removing the complexed target interfering compound that is bound to the binding agent.

In other embodiments, the method may employ a device of the disclosure. The method may comprise (a) obtaining a biological sample directly or indirectly from a subject; (b) adding the biological sample to a device to reduce a target interfering compound in a biological sample, the device comprising binding agent capable of binding a target within the biological sample; wherein binding of the target to the binding agent reduces the amount of target in the biological sample but does not significantly reduce the concentration of other analytes in the sample; and (c) incubating the biological sample in the device to permit the binding agent to bind the target; wherein the amount of target in the biological sample is reduced. The device may, in certain embodiments, comprise a receptacle having inner walls (or surfaces) defining an inner cavity to contain a biological sample. In some embodiments, the binding agent may be attached to one of the walls. Or, the device may comprise a binding agent attached to a solid support that is not part of the device but which may be added to the device (e.g., beads or other mobile solid surfaces).

In some embodiments, the binding agent binds the target with a K_{d} of about 10⁻¹⁰ M or less. In some embodiments, the target is reduced by at least 25-95% in the biological sample. In an embodiment, the binding agent is attached to the solid support via a linker.

In yet other embodiments, the invention comprises kits for using the devices and performing the methods disclosed herein.

### BRIEF DESCRIPTION OF THE FIGURES

The present invention may be better understood by referring to the following non-limiting figures.

FIG. 1 is a schematic illustrating a device having a plurality of binding agents attached to a solid support which bind a target interfering compound in the biological sample, but do not significantly bind other components of the sample.

FIG. 2 is a schematic illustrating a similar device as in FIG. 1 but in which the plurality of binding agents are attached to a mobile solid support such as a bead.

FIG. 3 shows results from a competitive immunoassay showing dose-dependent attenuation of biotin interference for measurement of free triiodothyronine (fT3).

FIG. 4 shows results from a two-site sandwich assay design showing dose-dependent attenuation of biotin interference for measurement of thyroid stimulating hormone (TSH).

### DETAILED DESCRIPTION

Titles or subtitles may be used in the specification for the convenience of a reader, which are not intended to influence the scope of the present invention.

It is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. It will be further understood that the endpoints of each of the ranges are significant both in relation to the other endpoint, and independently of the other endpoint. Thus, the endpoint of one range is combinable with the endpoint of another range. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples. It is to be understood, although not always explicitly stated, that all numerical designations may be preceded by the term “about.”

The term “agitate,” “agitating,” or “agitation” refers to creating motion or physically disturbing the contents. Contents (e.g. a liquid mixture in a container) are set in motion in a repeated or irregular manner. Agitation can include, without limitation, shaking, stirring, inverting, rotating, rolling, vibrating, spinning, or any combination thereof.

As used herein, an “analyte” refers to a molecule or compound that is being measured. In some embodiments, the analyte interacts with a binding agent. As described herein, the term “analyte” may refer to an atom, small molecule, nucleic acid, lipid, carbohydrate, oligosaccharide, protein, peptide, peptidomimetic, organic compounds and the like. An analyte may be an agonist, an antagonist, or a modulator. Or, an analyte may not have a biological effect.

The term “analytical test” refers to any test, procedure, experiment, and the like which analyzes the properties of a sample, particularly a biological sample. The test may analyze a component of the sample (e.g. an analyte in a biological fluid) for its properties (e.g. concentration), or may analyze the sample as a whole (e.g. the biological fluid) for its properties (e.g. viscosity). Analytical tests are typically performed under certain controlled conditions and provide repeatable results (e.g. within error margins) and information about a sample.

The term “antibody” includes monoclonal antibodies, polyclonal antibodies, synthetic antibodies and chimeric antibodies, e.g., generated by combinatorial mutagenesis and phage display. The term “antibody” also includes mimetics or peptidomimetics of antibodies. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like.

The term “antibody fragment” refers to any portion of the antibody that recognizes an epitope. Antibody fragments may be glycosylated. By way of non-limiting example, the antibody fragment may be a Fab fragment, a Fab′ fragment, a F(ab′)2 fragment, a Fv fragment, an rIgG fragment, a functional antibody fragment, single chain recombinant forms of the foregoing, and the like. F(ab′)2, Fab, Fab′ and Fv are antigen-binding fragments that can be generated from the variable region of IgG and IgM. They vary in size, valency, and Fc content. The fragments may be generated by any method, including expression of the constituents (e.g., heavy and light chain portions) by a cell or cell line, or multiple cells or cell lines. Preferably, the antibody fragment recognizes the epitope and contains a sufficient portion of an Fc region such that it is capable of binding an Fc receptor.

The term “biocompatible” means capable of being in direct contact with biological material without causing undesirable effects on biological material. Undesirable effects particularly include effects on the biological sample which interfere with or alter the results of analytical tests to be performed on the biological sample. For example, inert polymers may be biocompatible for lack of substantively modifying the biological sample. To the contrary, reactive agents such as peroxides, radicals, or highly active enzymes such as proteases, nucleases, etc., can significantly modify a portion or all of the biological sample. The term is intended to encompass some modification to the biological sample, for instance minor changes in amount or volume of the sample, inconsequential physical or chemical modification of components in the sample, etc. A change or modification is particularly inconsequential if it has substantially no effect on the outcome of an analytical test to be performed on the biological sample.

The term “biological sample” or “biological material” refers to any portion of a living or dead carbon-based organism. For instance, a biological sample may include cell debris, a single cell, group of cells, tissue, organs, fluid such as blood or urine, etc. The biological sample may be substantially unmodified or may be highly modified or processed (e.g. filtered, purified).

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps. Embodiments defined by each of these transition terms are within the scope of this invention.

As used herein, “concurrent” or “concurrently” refers to performing at least two steps at the same time or at approximately the same time. The term is also intended to include instances in which a first step initiates before a second step, but in which the second step initiates before the first step terminates.

The term “incubate” means to maintain under controlled conditions. Non-limiting controlled conditions include temperature, pressure, pH, duration, motion, and the like. Fluctuations in controlled conditions may occur so long as the fluctuations are by design. For example, a sample may be incubated for a period of time at 25° C., and by design (e.g. by intention) for a subsequent period of time at 37° C.

The term “interferes” or “interfering compound” or “target inteferring compound” or “target” refers to any molecule, compound, component, etc. which needs to be removed at least in part from a sample. In many cases, the target or target interfering compound can falsely alter the results of an analytical test. Alterations are false when the effect is to change the result in a way that is not reflective of reality. For instance, a sample may have a measurable property with an actual value of x, but an interfering compound causes analytical results to show the measurable property has a falsely increased value (e.g. 2×) or falsely decreased value (e.g. 0.5×).

“Optional” or “optionally” means that the subsequently described event or circumstance can or cannot occur, and that the description includes instances where the event or circumstance occurs and instances where it does not.

The term “reagent” means any substance (e.g. solid, liquid, or gas) added to a mixture, e.g. a biological fluid. The reagent can directly modify, chemically or otherwise, a component of the mixture (e.g. the reagent is a reactant in a chemical reaction). Alternatively, the reagent does not directly modify, chemically or otherwise, a component of the mixture (e.g. the reagent supplies the medium in which an interaction occurs).

The term “solid support” or “support” means a structure that provides a substrate onto which biomolecules may be bound. For example, a solid support may be immobilized on at least a portion of at least one inner wall of the inner cavity (e.g. an inner wall of a phlebotomy tube), or the solid support may be a mobile support, such as a bead.

The term “sterile,” “sterilize,” or “sterility” refers to the absence of viable organisms, microorganisms including, but not limited to bacteria, viruses, prions, spores, and other biological agents capable of replication. The term includes the presence of biological material and/or organisms but which are not viable or are otherwise incapable of replication. Thus, sterility does not require removal of nonviable biological material. For example, an object may be sterile even if the object contains killed organisms or killed biological material, so long as the organisms or biological material are not viable and not capable of self-replication.

The term “subject,” “individual,” “patient” and the like are used interchangeably herein and refer to any animal, or cells thereof whether in vitro or in situ, amenable to the methods described herein. In a preferred embodiment, the subject, individual, or patient is a mammal. In a particularly preferred embodiment, the subject, individual, or patient is a human.

The term “substantial” or “substantially” means all, nearly all, or, in some instances, mostly all of a thing, trait, property, action, etc. to which the term modifies. The term includes situations in which the comparative entity does not contain all of a thing, trait, property, action, etc., but does contain a sufficient amount of said thing, trait, property, action, etc. that it may be considered similar enough, for practical purposes under the circumstances, to be thought of as having all of the relevant thing, trait, property, action, etc. Thus, minor deviations which distinguish the comparative entity but do not amount to a substantive difference are inclusive in the term. The term excludes situations in the differences in a comparative entity's thing, trait, property, action, etc. are significant enough that the comparative entity would be considered substantively distinctive or distinguishable, such that the comparison is of little or no value. For example, situations in which substantially all of the bound target remains attached to the solid support means that, for all practical purposes under the circumstances of determining where the bound target is located, a very high percentage of bound target is attached to the solid support and thus, one may consider that essentially all of the bound target is attached to the solid support. However, a minor and inconsequential amount of bound target may not be attached to the solid support because, for instance, the linker was cleaved; but this difference does not result in any substantively distinctive or distinguishable amount of bound target not being attached to the solid support.

### Devices to Reduce Interfering Compounds

Disclosed are devices that use high affinity of immobilized biomolecules to bind target interfering compounds in a biological sample without binding other analytes of interest. Analytical tests performed on biological samples for analytes (i.e., molecules other than the interfering compound) treated in the device are less prone to interference by a contaminating target molecule(s).

For example, disclosed is device to reduce a target interfering compound in a biological sample comprising a binding agent attached to a solid support, the binding agent capable of binding a target within the biological sample, wherein binding of the target to the binding agent reduces the amount of target interfering compound in the biological sample

In some embodiments, the binding agent is attached to a solid support. In some embodiments, the device comprises a receptacle designed to collect and/or contain the biological sample and the binding agent is attached to an inner wall of the receptacle. In some embodiments, the binding agent is attached to the solid support via a linker.

Thus, in certain aspects, the invention comprises a device to reduce interfering compounds in a biological sample comprising a receptacle having inner walls defining an inner cavity to contain a biological sample; and a binding agent attached to a solid support, the binding agent capable of binding a target within the biological sample; wherein binding of the target to the binding agent reduces the amount of target in the biological sample but does not reduce the concentration of other molecules of interest (i.e., analytes) in the sample.

In some embodiments, the binding agent binds the target interfering compound with a dissociation constant (K_{d}) of about 10⁻¹⁰ M or less. In some embodiments, the binding agent comprises streptavidin, avidin, or an antibody. In some embodiments, the target comprises a target interfering compound. In some cases the target is biotin. Also, in some embodiments the biological sample comprises blood or plasma.

As noted herein, the device may include a receptacle in which a biological sample is placed. As used herein, the term “receptacle” means a container used to receive and/or store samples, particularly biological samples. The receptacle substantially maintains integrity of the biological sample and provides a reservoir for biological fluids. In certain embodiments, the receptacle may include flow-through devices in which entrance of the biological sample into the device is subsequently followed by exit through another opening, such as dual-opened tubing, capillaries, chromatography columns, and the like. Thus, the receptacle may be capable of containing the biological sample for more than a mere transient period of time. Containment within the receptacle provides a sealable environment in which the binding agent can bind the target. The receptacle can optionally be a hand-held device having portability. Increased ease of handling and portability may facilitate obtaining, transporting, storing, and analyzing biological samples contained therein.

The receptacle may contain at least one inner wall defining an inner cavity to contain a biological sample. As used herein, an “inner cavity” means a void within the receptacle in which a biological sample may be placed, contained, or stored. The at least one inner wall defining the inner cavity, in exemplary embodiments, may be leak-proof to prevent loss of biological fluids and/or reagents. A leak-proof inner cavity may also provide a chamber to store and mix the biological sample with other components. The at least one inner wall can optionally be coated, e.g. with a substantially inert polymer, to prevent leakage and/or undesirable interactions between the biological sample and the materials comprising the inner walls. As a non-limiting example, a phlebotomy tube contains at least one inner wall (the sides of the tubes) which define an inner cavity comprising the interior space within the tube. In some embodiments, the inner cavity can, but need not be, substantially devoid of additional matter, e.g. evacuated to remove gases and/or residual particles and liquids. In other embodiments, the inner cavity may be at least partially filled with matter prior to inserting the biological sample. For instance, the inner cavity may be partially filled with reagents, gels, beads, filters, solid supports, or other components.

The receptacle may include a means to insert a biological sample. In some embodiments, the receptacle can include one or more ports for receiving a biological sample. As used herein, a “port” is a space, region, or threshold through which a sample may pass to enter or exit the device. As an example, a port can simply be an opening in the device, exposing the inner cavity of the device to the exterior environment. As another example, the port can be a valve or valve system which permits selective entry of samples, material, or other matter.

The receptacle, in some embodiments, is optionally sealable. Sealing the device can enhance the storage and protection functions of the device and further avoid compromising the integrity of the biological sample. Seals may enclose or cover some or all of the one or more ports. For biological fluids, an exemplary seal is leak-proof In some embodiments, the seal can be liquid-tight to avoid leakage and/or be air-tight to substantially prevent the exchange of gases between the inner cavity and the exterior environment. The seal can be removable, for instance a cap, lid, or parafilm wrap. Alternatively, the seal may be fixed to the receptacle and capable of being partitioned or pierced to permit entry of a piercing device (e.g. needle), but which remains substantially sealed over the puncture upon removal of the piercing device by, for instance, returning to substantially the same shape and seal effectiveness, for instance a flexible septa, gel, or rubber stopper. More than one seal can be used to further insure against comprised integrity of the biological sample, for example placing a screw cap on a vial and wrapping the cap and vial with a parafilm or wax covering. Additionally, a portion or all of the device may be further covered or encapsulated to optimize protective measures for the biological sample. For instance, the sealed device may be placed in a secondary storage unit such as a box, rack, or sleeve.

A receptacle can optionally be a rigid container, e.g. a flask, bottle, or tube, or can be flexible, e.g. a bag. Non-limiting embodiments of receptacles include a tube, bag, syringe, catheter, flask, bottle, vial, capillary tube, pipette, pipette tip, plumbing tube, needle, microtiter plate, and multi-well collection device. Where the biological sample is a fluid such as blood, a particularly useful receptacle includes a phlebotomy tube. In exemplary embodiments, the receptacle is shatter-resistant to avoid spoiling or destroying the biological sample. Some commercially available receptacles include a Vacutainer blood collection tube from BD Biosciences and a Monoject blood collection tube from Covidien Ltd.-Medtronic.

The devices described herein may include a binding agent which binds a target in the biological sample. Binding kinetics are frequently expressed in terms of association and dissociation constants. As used herein, the dissociation constant (K_{d}) is an equilibrium constant that measures the propensity of a larger object to separate (dissociate) reversibly into smaller components, as when a complex separates into its component molecules. The dissociation constant is the inverse of the association constant (Kₐ), i.e., K_{d}=1/Kₐ. For a general reaction: AₓB_{y}↔→xA+yB, in which a complex AₓB_{y} breaks down into x A subunits and y B subunits, the dissociation constant Kd=([A]^{x}[B]^{y})/[AₓB_{y}], where [A], [B] and [AₓB_{y}], are the concentrations of A, B and the complex AₓB_{y}, respectively. Where x=y=1, K_{d} is such that [A]=K_{d} when [B]=[AB] or equivalently, [AB]/([B]+[AB])=½, such that K_{d} equals the concentration of free A at which half the total molecules of B are associated with A.

Affinity, concentration, ratio of binding agent to target, presence of inhibitors, properties of the medium, availability of other required binding partners such as cofactors, and other factors can affect binding kinetics. As an example, the presence of inhibitors which bind either the target or the binding agent will decrease the binding. Alternatively, the amount of binding agent within the device may be adjusted to, for instance, increase the saturation load, defined herein as the total amount of target in a biological sample that may be bound to the binding agent. Generally, increasing the amount of available binding agent in the presence of excess target will increase the saturation load. The surface area to volume ratio of the inner cavity may additionally influence binding. As the surface area to volume ratio increases, the propensity of liquid phase molecules to encounter the solid support within the fluid increases. One of ordinary skill in the art can calculate surface area to volume ratios for various basic shapes defined by the inner walls of the device. Optionally, the receptacle is elongated to optimize the surface area to volume ratio. Optionally, the surface area to volume ratio is about 1.0 or greater. Optionally, the surface area to volume ratio is at least about 1.1, 1.2, 1.3, 1.4, 1.5, 1.6, 1.7, 1.8, 1.9, or 2.0. Optionally, the surface area to volume ratio is greater than about 2.0.

Containment and handling of biological materials typically requires conditions of elevated safety, cleanliness, and/or sterility. Biological material often contains substrates to support microbial growth, which can drastically alter or destroy the biological sample. Because the receptacle directly contains the biological sample, the receptacle should exhibit these safe, clean, and/or sterile conditions. Optional safety features of the receptacle can include, but are not limited to, being manufactured of shatter-resistant or shatter-proof materials, being manufactured of temperature-resistant and chemically inert materials, inclusion of seals, e.g. lock-tight screw caps, which are not easily opened without intentional force, leak-proof design, and the like. The receptacle can be removed of dust, soiling, or other contaminants from the inner cavity. Optionally, the receptacle is substantially sterile for use in containing biological samples. Thus, materials which can be manufactured in a sterile process or can be autoclaved or chemically sterilized can advantageously comprise the receptacle.

The binding agent may be attached to a solid support, the binding agent capable of binding a target within the biological sample. As used herein, the term “binding agent” means a molecule that can specifically and selectively bind to a second (i.e., different) molecule of interest. The interaction may be non-covalent, for example, as a result of hydrogen-bonding, van der Waals interactions, or electrostatic or hydrophobic interactions, or it may be covalent. The binding agent generally has high affinity and specificity for the target interfering compound, but does not interact significantly with other components of the sample, such as an analyte of interest. Thus, the binding agent can bind and sequesters the interfering target in biological samples, thereby providing a mechanism to separate the target from the biological sample. After the target is bound by the binding agent, the remaining analytes in the biological sample can optionally be separated from the bound target, for example by removing the unbound portions of the biological sample to a second container.

A variety of binding agents may be used in the invention. The binding agent may be an affinity binding agent. The binding agent can optionally be a protein, peptide, lipid, carbohydrate, glycoprotein, nucleic acid, small molecule, chelator, or other molecule possessing the ability to specifically and selectively bind to a second molecule of interest. In some embodiments, the binding agent is a protein. Streptavidin is a protein originally discovered in Streptomyces avidinii which forms 56 kDa homotetramers. Streptavidin has high affinity and selectivity for biotin, binding up to four biotin molecules with a dissociation (K_{d}) of about 10⁻¹⁴ M. Avidin is a functionally analogous protein to streptavidin found in the eggs of birds, reptiles and amphibians which binds biotin with a K_{d} of about 10⁻¹⁵ M. Avidin is only 41% similar and 30% identical to streptavidin, but the secondary, tertiary, and quaternary structures are highly identical. See Laitenen et al., Cell. Mol. Life Sci., 63:2992-3017 (2006). At least 104 significantly homologous genes are known for streptavidin. See Dundas et al., Appl. Microbiol. Biotechnol., 97(21): 9343-9353 (2013), which is herein incorporated by reference in its entirety. Further, a wide array of streptavidin and avidin mutants have been engineered to tailor these proteins to specific uses. See id.; see also Laitinen et al. Trends in Biotechnology, 25(6): 269-277 (2007), which is herein incorporated by reference in its entirety.

The binding agent, in some embodiments, has very high affinity for the target. In some embodiments, the binding agent binds the target with a dissociation constant (K_{d}) of about 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, or 10⁻¹⁵ M or less. Optionally, the binding agent has high selectivity and affinity for biotin. Optionally, the binding agent is streptavidin or avidin, or any homologous protein thereof. As used herein, “homologous protein” means any protein, whether natural or engineered, having at least 50%, or 55%, or 60%, or 65%, or 70%, or 75%, or 80%, or 85%, or 90%, or 95% amino acid similarity to the reference protein (e.g. streptavidin or an antibody) and has a K_{d} of about 10⁻⁵M or less for the same target. As an example, a significantly homologous streptavidin protein includes any number of natural and engineered proteins which contain up to 1, 2, 3, 4, 5, 6, 7, 8, 9, 10, 15, 20, 25, 30, 40, or 50% amino acid similarity to streptavidin and binds a target, for instance biotin, with a K_{d} of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or less.

Optionally, the binding agent is an antibody or antibody fragment. Antibodies from a number of sources are compatible with the present invention. For instance, the antibody can be, without limitation, a monoclonal or polyclonal antibody or antibody fragment from a murine, porcine, bovine, rabbit, chicken, goat, pig, human, humanized, or chimeric source. As an example, commercially available anti-biotin antibodies can be obtained from Thermo Fisher Scientific. One of skill in the art would appreciate that antibodies and antibody fragments tolerate many amino acid mutations such as substitutions, additions, and deletions, yet retain high selectivity and affinity for cognate ligands. As an example, a technique known as CDR-grafting involves grafting up to all six of the complementarity-determining regions (CDRs) of a donor antibody onto an acceptor antibody from a different animal species.

The binding agent is capable of binding the target interfering molecule while both are within or in direct contact with a biological sample. The binding agent optionally is biocompatible with the biological sample and thus, avoids destroying, spoiling, or otherwise significantly reducing the integrity of the biological sample. The binding agent, in some embodiments, does not bind to or significantly reduce the concentration of other components in the biological sample. It is, however, appreciated that the binding agent may bind or interact with other components in addition to the target. In such circumstances, the biological sample is compatible with the present disclosure so long as the reduction in concentration of components other than the target does not significantly interfere with the results of an analytical test to be performed on the biological sample. In some embodiments, a plurality of binding agents are attached to the solid support.

The binding agent is capable of binding a target within the biological sample, wherein binding of the target to the binding agent reduces the amount of target in the biological sample. As used herein, the term “target” means any component present in a biological sample, including, but not limited, to an atom, molecule, compound, polymer, or biological material such as small molecules, proteins, lipids, carbohydrates, glycoproteins, nucleic acids, cells, or cell debris. The target can be any natural, synthetic, or engineered component in the biological sample. Optionally, the target can be within the biological sample at the time the biological sample is obtained from a subject, or alternatively, added to the biological sample after it is obtained from the subject. Optionally, the target interferes with at least one analytical test to be performed on the biological sample or imparts undesirable properties to the biological sample. For instance, certain lipids can alter the results obtained in serum bilirubin tests. Additionally, high blood levels of biotin interfere with a range of laboratory tests, particularly immunoassays. Reduction of the amount of the target in the biological sample thus optionally improves the quantification, accuracy, reliability, sensitivity, and/or repeatability of an analytical test to be performed on the biological sample.

The size of the target is not particularly limited so long as the target can stably bind the binding agent. Optionally, the target has a molecular weight of about 1,000 kDa or less, 500 kDa or less, 400 kDa or less, 300 kDa or less, 200 kDa or less, 100 kDa or less, 50 kDa or less, 25 kDa or less, 10 kDa or less, or 1 kDa or less. The target can also be a small molecule having a low molecular weight. For instance, the target can have a molecular weight of about 0.5 kDa or less. In some embodiments, the target contains a ureido ring and/or a terminal carboxyl group. In some embodiments, the target is biotin or a biotin-conjugated molecule. As used herein, the term “biotin” refers to a compound containing a ureido ring fused with a tetrahydrothiophene ring and having a terminal carboxyl group, having a general chemical formula of C₁₀H₁₆N₂O₃S. Formula 1 shows the chemical structure of biotin:

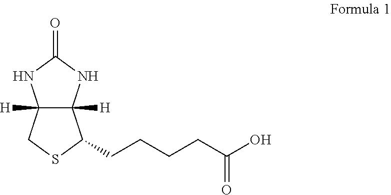 [SMILES: O=C(O)CCCC[C@@H]1SC[C@@H]2NC(=O)N[C@@H]21]

Biotin is also known as vitamin B7, vitamin H or coenzyme R. It is appreciated that the term “biotin” includes biotin-like molecules having minor changes in the base molecule shown in Formula 1 but which significantly resemble the base molecule. As an example, the terminal carboxyl may be deprotonated or amidated. The target, e.g. biotin, can be dissolved in the biological sample as a free molecule. Alternatively, the target, e.g. biotin, can be covalently conjugated to other molecules such as, without limitation, peptides, proteins, small molecules, and nucleic acids.

The target may be present in the biological sample at normal physiological levels, at reduced levels, or at elevated levels. This wide range of target levels reflects that even sub-physiological levels of certain targets may still interfere with an analytical test. The invention is particularly useful to reduce the amount of elevated levels of the target.

In some instances, the target may interfere with an analytical test to be performed on the biological sample. For instance, hemolysis tests can be affected by ammonia, amylase, bilirubin, carotene, ceruloplasmin, digixon, folic acid, insulin, testosterone, certain triglycerides, alkaline phosphatase, creatine phosphokinase, folate, LDH, AST, ALT, chloride, lithium, magnesium, phosphorus, sodium, and other compounds; albumin tests can be affected by aspirin, heparin, and penicillins; alkaline phosphatase tests can be affected by anticoagulants, estrogens, gentamicin, and albumin; amylase tests can be affected by oxalate, citrate, opiates, and oral contraceptives; bilirubin tests can be affected by daylight, barbiturates, and drugs having liver toxicity or causing cholestasis; calcium tests can be affected by diuretics, phenytoin, antacids, and vitamin D; cholesterol tests can be affected by neomycin, bilirubin, and corticosteroids; creatinine tests can be affected by acetoacetic acid, acetone, and ascorbic acid; glucose tests can be affected by ascorbic acid; occult blood tests can be affected by ascorbic acid, meat fibers, and aspirin; ionized magnesium tests can be affected by silicon, heparin, and zinc [see Ritter et al, Scand. J. Clin. Lab. Invest. Suppl., 224:275-280 (1996)]; and Limulus amebocyte lysate (LAL) endotoxin tests can be affected by pyrogens, plasma, and blood amidases [see Hurley, Clin. Microbial. Rev., 8(2):268-292 (1995)]. Analysis of cardiac markers in emergency room patients is also susceptible to elevated biotin levels. Biotin-mediated interference may also cause prescription of inaccurate dosages; for instance patients may have their thyroid medication titrated improperly. As a result of biotin's high affinity for streptavidin (K_{d} of about 10⁻¹⁴ M), a wide range of binding assays and separations techniques are based on the unique properties of the biotin-streptavidin interaction. Excess free biotin substantially interferes with biotin-streptavidin binding assays, particularly assays using biotin conjugated molecules.

The amount of the target may be artificially elevated by, for instance, diet, genetic predisposition, medical treatment, acute or prolonged environmental exposure, and/or supplementation. As a non-limiting example, aggressive advertising campaigns in the health and beauty markets have fueled biotin supplementation in recent years. Although the Recommended Daily Intake (RDI) of biotin is only 30 μg per day, commercially available biotin supplements in the form of pills, capsules, gels, etc. contain up to 10 mg or more of biotin. Average biotin levels in normal human blood serum are about 57-2460 pg/mL for pediatric patients below twelve years old, and about 221-3004 pg/mL for adult patients twelve years and older. However, consumers of biotin supplements may consume large quantities of biotin up to 3, 5, 10, 30, 50, 100, 200, 300 mg, or more, per day.

Metabolism and excretion rates affect the degree of interference a target has on laboratory and diagnostic tests. In the non-limiting case of biotin, the in vivo half-life is approximately two hours. While interfering levels of minor supplementation (<1 mg biotin) should clear the body within several hours, megadoses of biotin, e.g. about 30 mg, can be interfering for up to about 24 hours. As such, abstaining from supplementing with megadoses of biotin for at least about two days prior to obtaining a biological sample is advisable.

In some embodiments, the target is present in a biological sample from a pediatric patient at greater than about 2,460 pg/mL or in a biological sample from an adult patient at greater than about 3,004 pg/mL. In some embodiments, the target is present in a biological sample from about 3,000 pg/mL to about 30,000,000 pg/mL, from about 5,000 pg/mL to about 10,000,000 pg/mL, from about 10,000 pg/mL to about 1,000,000 pg/mL, from about 50,000 pg/mL to about 500,000 pg/mL, or from about 100,000 pg/mL to about 250,000 pg/mL. In some embodiments, the target is a non-natural contaminant. In some embodiments, the target is present naturally in vivo, but in quantities greater than the average known for the species of subject from which the biological sample is drawn. Optionally, the target is present at elevated levels up to about 5, 10, 50, 100, 500, 1,000, 5,000, or 10,000 times the average biological level of the target for the species of subject.

Binding agent-mediated reduction of the amount of the target in the biological sample can optionally be in advance of a further step, e.g. an analytical test. In embodiments in which the target interferes with an analytical test, target bound to the binding agent is removed from the biological sample, wherein the amount of free, interfering target remaining in the biological sample is reduced. Advantageously, the amount of free, interfering target remaining in the biological sample can be reduced below a threshold concentration which interferes with the analytical test to be performed on the biological sample. In some embodiments, the concentration of the target in the biological sample can be reduced by at least 5, 10, 50, 100, 500, 1,000, 5,000, or 10,000 times. In some embodiments, the concentration of the target in the biological sample can be reduced to 1,000,000 pg/mL or less, 500,000 pg/mL or less, 100,000 pg/mL or less, 50,000 pg/mL or less, 10,000 pg/mL or less, or 5,000 pg/mL or less.

The herein disclosed device may include a solid support to which the biological agent is attached. The solid support may be a construct to which molecules may be attached and become separable from the biological sample. As with other components in the device, the solid support is substantially chemically inert, biocompatible, sterile, and resists modifying the biological sample in ways other than its role in binding the target.

Composition and positioning of the solid support can vary. The solid support may optionally be attached to and/or immobilized on the device. For instance, the solid support can be all or a portion of at least one inner wall of the inner cavity. In such an embodiment, the biological agent is directly or indirectly attached to the inner walls. Or, the solid support may be a filter, through which the sample is passed to remove the potentially interfering target molecules. In some embodiments, the solid support can be mobile. For instance, the solid support can be added to the inner cavity prior to, concurrent with, or subsequent to addition of the biological support to the inner cavity. Separate mobile solid supports facilitates interchangeability of solid supports, and thus binding agents, with the receptacle. In some embodiments, the solid support is a fiber, filament, tubule, bead, mesh, capillary, cartridge, membrane, resin, matrix, or any combination thereof. Thus, the device optionally comprises a plurality of solid supports. As a non-limiting example, beads and resins are frequently used in many batch and analytical separation techniques. Beads and resins may be added directly to the receptacle, mixed with the biological sample, and easily separated from the biological sample. Beads may be comprised of a resin, polymer such as polyvinyl, agarose, silica, magnetite, polyacrylamide, and other compatible materials. Beads and resins may be coated with a wide variety of binding agents. Non-limiting commercially available coated beads include Streptavidin-coupled Dynabeads® available from ThermoFisher Scientific, Streptavidin MicroBeads available from MACS Miltenyl Biotec, and Sphero™ avidin-coated polystyrene particles from Spherotech Inc.

As noted herein, the biological agent may be attached directly to the solid support. The attachment between the biological agent and a solid support includes any stable attachment between molecules including, but not limited to, covalent bonds, ionic bonds, hydrogen bonds, dipole-dipole interactions, hydrophobic interaction, van der walls forces, and the like.

Additionally and/or alternatively, the biological agent is attached to the solid support via an optional linker. The linker functions as a structural intermediary in the attachment of a binding agent to the solid support. As used herein, the term “linker” means any molecule, compound, oligomer, polymer, homomer, heteromer, matrix, and the like which attaches, directly or indirectly, the binding agent to a solid support. Alternatively, the linker can be a bond, e.g. a covalent bond. Because the binding agent can be attached to a linker which, in turn, is attached to a solid support, binding of the target to the binding agent sequesters the target near the solid support. The attachment between a linker and a solid support, or between a linker and a binding agent, includes any stable attachment between molecules including, but not limited to, covalent bonds, ionic bonds, hydrogen bonds and the like. The linker can optionally be synthetic, biological, or any combination thereof.

Optionally, the linker can be, but is not limited to, an adhesive, coating, polymer, protein, peptide, nucleic acid, or other such molecular linkers. The linker optionally can include at least one functional group. The at least one functional group can optionally attach the linker to a solid support, a binding agent, or other compounds. In some embodiments, the linker can include two or more functional groups. The two or more functional groups can be different functional groups, which can facilitate attachment of the linker to the solid support in one step and attachment of the linker to a binding agent in a second step. Optionally, the two or more functional groups can be the same functional groups or different functional groups which have the same or similar chemistries, which can facilitate attachment of the linker to the solid support and the binding agent in one step. In some embodiments, the linker can be polyethylene glycol (PEG) and/or polypropylene glycol (PPG). As a non-limiting example, a wide range of PEG molecules may be coupled to a wide range of polypeptides. Coupling chemistries for PEG-protein conjugates (e.g. pegylated proteins) are well known in the art. For instance, an active ester of a carboxylic acid-functionalized PEG is readily coupled to a primary amine of a protein to form an amide bond. Protein pegylation has been extensively reviewed. See, e.g., Roberts et. al., Advanced Drug Delivery Reviews, 54: 459-476 (2002), incorporated herein by reference in its entirety.

Positioning of the optional linker determines positioning of the binding agent. In some instances, it may be desirable to maximize the target-sequestering effects of the binding agent. Thus, the linker may optionally be a molecular layer substantially coating or covering the entire solid support. In other circumstances, it may be desirable to localize the target-sequestering effects of the binding agent. Thus, the linker may optionally be a molecular layer substantially coating or covering at least a portion of the solid support. For instance, the linker may coat or cover at least a portion or all of the inner walls of the inner cavity, fibers or polymeric beads.

The linker may be exposed to biological samples and, as such, the linker may optionally be stable in the presence of biological material. Biological samples often contain components which may degrade, sequester, inactivate, or otherwise modify substrates. For instance, blood can contain substantial levels of globulins, albumins, immunomodulatory factors, enzymes such as kinases, phosphatases, aminotransferases, and oxidases, and the like. Linker resistance to modifying molecules, particularly cleaving enzymes such as proteases, lipases and nucleases, avoids release of binding agent in the sample which can significantly inhibit the ability to separate the target from the biological sample. The linker may optionally be stable in the presence of one or more reagents added to the biological material for similar reasons. In other embodiments, the linker may, by design, be susceptible to cleavage or degradation by one or more reagents to release the binding agent, the target, or both. To avoid adverse effects on the biological sample, the linker may optionally be biocompatible.

Optionally, the device may be further equipped with a filter to facilitate separation of bound target and the biological sample. A filter substantially permeable to the biological sample but substantially impermeable to the solid support, or to which the binding agent has been attached, can permit easy entrance and exit of the biological sample into the receptacle but retain bound target and/or the solid support when the biological sample is removed. As an example, a fluidic biological sample may simply be poured out of the device and into a new container while the filter retains beads, and hence bound target, within the inner cavity of the receptacle.

The biological sample used in the present invention is typically a biological fluid. The biological sample may be present in amounts ranging from microliter amounts to liter amounts, e.g. from about 1 μL to about 1 liter. Optionally, the biological sample may be present in an amount from about 5 μL to about 500 mL, from about 10 μL to about 100 mL, from about 100 μL to about 80 mL, from about 1 mL to about 50 mL, or from about 10 mL to about 40 mL. Non-limiting examples of biological fluids include blood, plasma, lymph, saliva, sputum, exudate, urine, bile, mucus, semen, amniotic fluid, chime, pleural fluid, breast milk, pericardial fluid, peritoneal fluid, lacrimal fluid, synovial fluid, serous fluid, gastric acids, cerebrospinal fluid, sebum, interstitial fluid, and diaphoresis fluid. In an exemplary embodiment, the biological sample is blood and/or plasma.

Upon addition to the device, the biological sample is exposed to the binding agent. Thus, the concentration or amount of free target in the biological sample is reduced over time as the target binds the binding agent. Optionally, the biological sample may be mixed with a reagent in the inner cavity of the receptacle. In some embodiments, the reagent measures, detects, or provides information on an analyte in the biological sample. In some embodiments, the reagent facilitates, enhances, expedites, or otherwise increases the binding of the target to the binding agent.

### Methods to Reduce Interfering Compounds

In certain aspects, the herein disclosed methods utilize the high affinity of immobilized biomolecules to bind target compounds in a biological sample within a receptacle designed to collect and/or contain the biological sample. The methods are capable of reducing unbound target in the biological sample by at least 25-95%. The methods are particularly useful to pre-treat a biological sample to reduce the amount of a compound, e.g., biotin, which interferes with an analytical test to be performed on the biological sample.

For example, disclosed is a method of reducing interfering compounds in a biological sample comprising (a) obtaining a biological sample directly or indirectly from a subject; (b) adding a binding agent capable of binding a target within the biological sample; wherein binding of the target to the binding agent reduces the amount of target in the biological sample; and (c) incubating the biological sample in the device to permit the binding agent to bind the target; wherein the amount of target in the biological sample is reduced. In some embodiments, binding agent attached to a solid support. In some embodiments, the binding agent is attached to the solid support via a linker.

In some embodiments, the biological sample is added to a device, such as the devices disclosed herein, comprising a binding agent that binds the target. In some embodiments, binding agent is attached to a solid support. In some embodiments, the binding agent is attached to the solid support via a linker. In some embodiments, the device may comprise a receptacle having inner walls defining an inner cavity to contain a biological sample and the binding agent is attached to the inner walls of the container. Or, the solid support may be a mobile support and/or a filter as disclosed herein.

In some embodiments, the binding agent binds the target with a dissociation constant (K_{d}) of about 10⁻¹⁰ M or less. In some embodiments, the target is reduced by at least 25-95% in the biological sample.

In some embodiments, the target is a target interfering compound. For example, the target may be biotin and the binding agent comprises streptavidin, avidin, or an anti-biotin antibody.

The step of obtaining a biological sample may proceed by a variety of methods appropriate for the type of biological sample to be obtained. Thus, the methods used to obtain a sample depend in part on the specific biological sample. The biological sample used in the present invention is typically a biological fluid. Non-limiting examples of biological fluids include blood, plasma, lymph, saliva, sputum, exudate, urine, bile, mucus, semen, amniotic fluid, chime, pleural fluid, breast milk, pericardial fluid, peritoneal fluid, lacrimal fluid, synovial fluid, serous fluid, gastric acids, cerebrospinal fluid, sebum, interstitial fluid, diaphoresis fluid, and the like. Thus, non-limiting methods to obtain a sample include, but are not limited to, blood and/or plasma draw (e.g. veripuncture), blood donation and other phlebotomy techniques, puncture methods such as lumbar puncture or arthrocentesis, swab, biopsy, passive drool using e.g. a saliva collection aid and cyrovial available from Salimetrics, LLC, placement of sample into a container by the subject as in e.g. urine, semen, or breast milk donation, and other methods.

The term “directly or indirectly” as used herein in relation to the obtaining step refers to a method performer's direct obtaining of a sample from a subject, and a method performer's indirect obtaining of a sample obtained by another individual from the subject. In the former, the method performer performs the actual biological sample collection method, e.g. venipuncture. In the latter, the method performer receives the biological sample collected by another individual, e.g. a phlebotomist, who performs the actual biological sample collection method, e.g. venipuncture. Further, any number of individuals, agencies, machines, etc. may handle, store, modify, transport, divide, or perform other actions on or with the biological sample between the actual collection thereof, e.g. venipuncture, and the receipt or obtaining of the biological sample by the method performer. At least all of the above scenarios are captured by the term “directly or indirectly” as used herein.

Depending on the type of biological sample, there can be a wide range of time between the obtaining step and the adding step. Optionally, the biological sample can be freshly obtained, e.g. within seconds or minutes of adding the biological sample to the device. Alternatively, the biological sample may be stored prior to addition to the device, e.g. in cryogenic storage conditions. Alternatively, the biological sample may be processed prior to addition to the device, e.g. in one or more analytical tests, separation techniques, filtering techniques, etc. Optionally, the obtaining and adding steps may proceed concurrently. Thus, a biological sample may be obtained directly or indirectly from a subject and added to the inner cavity of the device in a substantially concurrent step. Concurrent obtaining and adding steps may be performed when, for example, a subject directly places the biological sample, e.g. urine, into the device. Another example includes the use of phlebotomy collection kits attached to the device. A hollow needle may be inserted into the subject, drawing blood through phlebotomy tubes and into the device. As such, the device may be modified to meet compatibility requirements of various collection devices.

The biological sample may be obtained in amounts ranging from microliter amounts to liter amounts, e.g. from about 1 μL to about 1 liter. As examples, finger-prick insulin-measuring devices can obtain as little as about 3 μL of blood, whereas blood donations can collect about 0.5 liters of blood from a donor. Optionally, the biological sample can be obtained in an amount from about 5 μL to about 500 mL, from about 10 μL to about 100 mL, from about 100 μL to about 80 mL, from about 500 μL to about 75 mL, from about 1 mL to about 50 mL, from about 1 mL to about 20 mL, or from about 1 mL to about 10 mL

Although supplementation with megadoses of biotin can increase biotin levels in the blood to levels which affect the accuracy of analytical tests, biotin has an in vivo half-life of approximately two hours. Avoiding megadoses of biotin supplementation for at least 48 hours or more can significantly reduce the blood concentration of biotin in a subject. However, an at least 48 hour waiting period prior to obtaining a sample presents practical problems. For instance, a subject may be presently ready to donate a biological sample (e.g. the subject is present at a medical clinic). Thus, one advantage of the herein disclosed invention is that it permits obtaining a biological sample from a subject regardless of whether the subject was recently exposed to high levels of a target (e.g. ingested megadoses of biotin).

The adding step typically proceeds by placing the biological sample into the device through one or more ports of the receptacle. The sample may be added by a variety of means such as, but not limited to, flowing, dripping, injection, pouring, pipetting, dispensing, etc. A sufficient amount of biological sample is positioned within the receptacle for the binding agent to bind the target. Thus, for fluidic biological samples, an amount sufficient to submerge at least a portion and preferably all of the solid support can optionally be added.

The adding step can optionally conclude with a sealing step in which the receptacle is closed and sealed. Optionally, the adding and sealing steps can be repeated two or more times until a desired amount of biological sample is disposed within the receptacle.

The biological sample is incubated in the device to permit the binding agent to bind the target, thereby reducing the amount of target in the biological sample. The incubation step can proceed for a range of time periods depending in part on parameters such as affinity between the binding agent and the target, desired binding saturation, molar amounts of target and/or binding agent, presence of binding inhibitors, fluid viscosity, pH, ionic strength, temperature, etc. As an example, even very high amounts of biotin, e.g. up to 30,000,000 pg/mL paired with a binding agent having a dissociation constant for biotin of about 10⁻¹⁴M or less, e.g. streptavidin or avidin, may require an incubation step optionally proceeding for a period of time in the order of seconds or minutes. However, situations in which, for example, the target and binding agent have lower affinities or in which the target must be reduced to extremely low levels, the incubating step can proceed for longer periods of times, e.g. hours or days. In some embodiments, the biological sample is incubated in the device from about 1 second to about 1 week, from about 30 seconds to about 48 hours, from about 1 minute to about 24 hours, from about 2 minutes to about 12 hours, from about 5 minutes to about 10 hours, from about 10 minutes to about 5 hours, or from about 15 minutes to about 2 hours. One of skill in the art would appreciate that binding of the target to the binding agent can begin essentially instantaneously upon addition of the biological fluid to the device. Thus, the separate steps of adding and incubating are not to be interpreted as limiting binding to occur only during the incubating step alone.

During the incubation step, the binding agent optionally binds the target with a K_{d} of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or less. Preferably, the binding agent binds the target with a K_{d} of about 10¹⁰ M or less, more preferably with a K_{d} of about 10⁻¹⁴ M or less. In some embodiments, the target can be biotin. In some embodiments, the binding agent can be streptavidin or avidin. In some embodiments, the binding agent can be an antibody, e.g. an anti-biotin antibody.

Conditions for the incubating step can influence the rate of binding between the target and the binding agent. The receptacle may optionally be sealed during the incubation step to control for particular conditions the biological sample is subjected to. As an example, the viscosity of blood is inversely proportional to temperature. Streptavidin-biotin interactions are stable over a range of temperatures, but the interaction may be broken in nonionic aqueous solutions at temperatures above 70° C. See Holmberg et al., Electrophoresis, 26(3): 501-510 (2005), which is incorporated by reference herein. Thus, the biological sample can optionally be incubated in the device at a temperature up to about 70° C., up to about 60° C., up to about 50° C., up to about 40° C., up to about 30° C., up to about 20° C., or up to about 10° C. In some embodiments, the biological sample is incubated in the device at a temperature from about 10° C. to about 40° C., from about 15° C. to about 30° C., from about 18° C. to about 25° C., from about 19° C. to about 23° C., or from about 20° C. to about 22° C. In some embodiments, the biological sample is incubated in the device at about room temperature. As another example, contact between the biological sample and the solid support can be facilitated by agitating the device, particularly where the solid support is not completely submerged by the biological sample. Thus, the incubating step may optionally further comprise agitating the device to, for example, coat the solid support with the biological fluid. Agitation can include, without limitation, shaking, stirring, inverting, rotating, rolling, vibrating, spinning, or any combination thereof.

The method can optionally further comprise adding a reagent to the device. Optionally, the biological sample may be mixed with a reagent in the inner cavity of the receptacle. In some embodiments, the reagent measures, detects, or provides information on an analyte in the biological sample. In some embodiments, the reagent facilitates, enhances, expedites, or otherwise increases the binding of the target to the binding agent. The reagent can optionally be a reagent used in an analytical test in which the target interferes with the accuracy thereof. In such embodiments, the amount of free target in the biological sample must first be reduced below a threshold which does not substantially interfere with the accuracy of the analytical test.

The method can optionally further comprise separating at least a portion of the biological sample from the solid support. Separation can result in a biological sample having a reduced amount of free, soluble target and is substantially free of bound target. In some embodiments, substantially all of the target bound to the binding agent remains in the inner cavity of the device upon removing the biological sample. Separation can avoid interference reactions which may still occur between test reagents and bound target. Separation may also result in a processed biological sample which can be fed into other containers, devices, machines, etc. for further processing, analysis, experimentation, and the like.

Separation of the biological sample from the bound target may be performed by a variety of methods. In embodiments in which at least a portion of at least one inner wall is the solid support, separation methods are relatively straight forward. For instance, the biological fluid can be decanted from the device or, alternatively, can be pipetted out of the device. Additional separation steps may be used in embodiments in which the solid support comprises mobile solid supports such as a fiber, filament, tubule, bead, mesh, capillary, cartridge, membrane, resin, or matrix. For instance, beads can be sedimented, or collected at the bottom of the inner cavity. By positioning the solid support at the bottom of the inner cavity, withdrawal devices such as pipettes can be used to remove biological sample from regions above the sedimented solid support. Sedimenting can be accelerated by several ways, including centrifugation or by magnetism in the case of magnetized beads. In other embodiments, a filter permeable to the biological agent but not to the solid support can be used to remove the poured biological agent but retain the solid support.

As discussed, use of biological samples can require sterile or near-sterile conditions, particularly where the method to obtain the sample is invasive (e.g. insertion of a needle) and where the accuracy of an analytical test is sensitive to spoilage. Thus, any one or more of the obtaining, adding, and incubating steps, as well as any one or more of handling, storage, and other steps may optionally be performed using sterile or substantially sterile conditions.

The methods described herein reduce the amount of the target in the biological sample. In some embodiments, the target is reduced by at least 10%, 20%, 30%, 40%, 50%, 60%, 70%, 80%, 90%, or 100%. In some embodiments, the target is reduced by at least 25%, 50%, 75%, 95%, 96%, 97%, 98%, or 99%. By “reduced amounts of target,” it is meant that the amount of free target is reduced in the biological sample. thus, a reduced amount of target occurs at least when the target in the biological sample binds the binding agent, regardless whether the biological sample is separated from the bound target.

### Kits to Reduce Interfering Compounds

Also disclosed are kits to use the devices and perform the methods described herein.

In one embodiment, the kit may comprise a device to reduce target interfering compounds in a biological sample comprising a binding agent capable of binding a target within the biological sample, wherein binding of the target to the binding agent reduces the amount of target in the biological sample; and instructions for use

In one embodiment, the binding agent is attached a solid support. For example, in one embodiment, the kit may comprise a device to reduce interfering compounds in a biological sample comprising a receptacle having inner walls defining an inner cavity to contain a biological sample; and a binding agent attached to a solid support, the binding agent capable of binding a target within the biological sample; wherein binding of the target to the binding agent reduces the amount of target in the biological sample; and instructions for use. In one embodiment, the binding agent may be attached to an inner wall of the receptacle. Or in other embodiments, The binding agent may be attached to a mobile solid support. In some embodiments, the binding agent is attached to the solid support via a linker.

In some embodiments, the binding agent binds the target with a dissociation constant (K_{d}) of about 10⁻¹⁰ M or less. In some embodiments, the binding agent comprises streptavidin, avidin, or an antibody. In some embodiments, the target comprises biotin. The biological sample may comprise blood or plasma or other biological samples.

The kit can include any of the herein disclosed devices and can be used to perform any of the herein disclosed methods. As such, the kit can include a binding agent which optionally binds the target with a K_{d} of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or less. Preferably, the binding agent binds the target with a K_{d} of about 10⁻¹⁰ M or less, more preferably with a K_{d} of about 10⁻¹⁴ M or less. In some embodiments, the kit can include a binding agent which binds biotin. In some embodiments, the kit can include streptavidin or avidin as the binding agent. In some embodiments, the kit can include an antibody, e.g. an anti-biotin antibody, as the binding agent.

The kit can optionally include a receptacle pre-loaded with a particular binding agent attached to an immobile solid support in the device. Optionally, the kit includes one or more mobile solid supports which may be added to the device depending on the desired target to be reduced in the biological sample. Thus, the kit can include a fiber, filament, tubule, bead, mesh, capillary, cartridge, membrane, resin, matrix, or any combination thereof which can be added into the device. Optionally, the mobile solid supports may have interchangeable binding agents which may be attached, or separate solid supports which may be fixed with separate binding agents. Optionally, the kit can include one or more linkers which may be compatible with various binding agents and/or solid supports. The optional linker can be first attached to a solid support and subsequently attached to a binding agent. Alternatively, the optional linker can be first attached to a binding agent and subsequently attached to a solid support. Alternatively, the optional linker can be attached to a binding agent and a solid support in the same step. Thus, interchangeable solid supports with different binding agents permits an array of binding agents which can be selected for use in the device. Optionally, the kit further comprises filters which are permeable to the biological sample but impermeable to the solid support.

Optionally, the kit further comprises reagents to perform an analytical test on the biological sample. In such embodiments, the kit further comprises instructions for interpreting results of the analytical test.

Optionally, the kit further comprises reagents to strip or remove bound target. Stripping reagents can vary depending on the target to be stripped, but can include strong organic solvents, acids, bases, denaturants, and other reagents. Further, the device may be sterilized one or more times. Stripped and sterilized devices may optionally be reused. Such stripped devices may require reloading a binding agent provided in the kit, which may have been removed or have lost function after exposure to stripping reagents and sterilization methods.

### DETAILED DESCRIPTION OF THE FIGURE

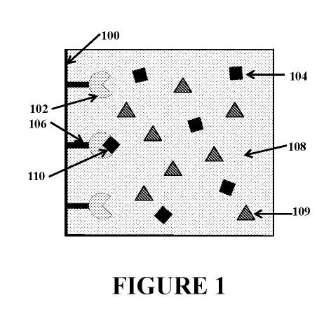

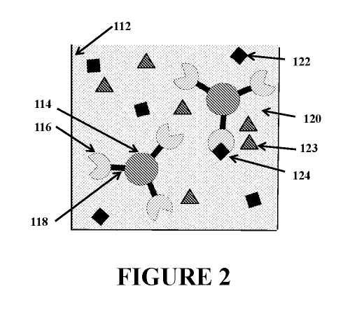

FIGS. 1 and 2 provide schematics illustrating embodiments of the device to reduce interfering compounds in biological samples. A receptacle having inner walls (100) defining an inner cavity is zoomed in on. As used herein, a wall includes any surface of a receptacle including sides, bottom and top. In the embodiment shown in FIG. 1, the inner wall also serves as the solid support. A binding agent (102) is attached to the solid support and is capable of binding a target (104), wherein the target is a molecule (e.g., biotin) that can interfere with an assay for a molecule or compound of interest (i.e., an analyte) (109) e.g., thyroid stimulating hormone (TSH), being measured. FIG. 1 further shows optional features including a plurality of binding agents, any one of which can be attached to the solid support (100) by an optional linker (106). A biological sample (108), shown here as a biological fluid having a plurality of free, unbound interfering targets (104) and analytes of interest (109), is contained within the device. The binding agent specifically and selectively binds the interfering target (!04), resulting in a stable binding complex of bound target (110) while not binding the analyte of interest (109).

FIG. 2 shows an alternative embodiment in which, rather than having the binding agent (116) attached to an inner surface (112), a mobile solid support (114) such as a bead is used. The binding agent (116) can optionally be attached to the solid support (114) via a linker (118). Upon adding a biological sample (120) containing free, unbound interfering target (122) and the analyte of interest (123), the binding agent binds the target, resulting in a stable binding complex of bound target (124), whereas the analyte of interest (123) is not bound.

Also, disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Various non-limiting embodiments of the invention are described below.

A1. In certain embodiments, disclosed is device to reduce interfering compounds in a biological sample comprising: a receptacle having inner walls defining an inner cavity to contain a biological sample; and a binding agent attached to a solid support, the binding agent capable of binding a target within the biological sample; wherein binding of the target to the binding agent reduces the amount of target in the biological sample.

A2. The device of embodiment A1, wherein the binding agent is attached to the solid support via a linker.

A3. The device any one of the preceding embodiments, wherein the binding agent binds the target with a dissociation constant (K_{d}) of about 10⁻¹⁰ M or less.

A4. The device of any one of the preceding embodiments, wherein the binding agent binds the target with a dissociation constant (K_{d}) of about 10⁻¹⁴ M or less.

A5. The device of any one of the preceding embodiments, wherein the biological sample comprises blood, plasma, or any combination thereof.

A6. The device of any one of the preceding embodiments, wherein the binding agent comprises streptavidin or avidin.

A7. The device of any one of the preceding embodiments, wherein the binding agent is an antibody.

A8. The device of any one of the preceding embodiments, wherein the target interferes with at least one analytical test to be performed on the biological sample.

A9. The device of any one of the preceding embodiments, wherein the target comprises biotin.

A10. The device of any one of the preceding embodiments, wherein the receptacle comprises a tube, bag, syringe, catheter, flask, bottle, vial, capillary tube, pipette, pipette tip, plumbing tube, needle, microtiter plate, multi-well collection device, or any combination thereof.

A11. The device of any one of the preceding embodiments, wherein at least a portion of at least one inner wall of the inner cavity comprises the solid support.

A12. The device of any one of the preceding embodiments, wherein the solid support is a fiber, filament, tubule, bead, mesh, capillary, cartridge, membrane, resin, matrix, or any combination thereof.

A13. The device of any one of the preceding embodiments, wherein the device comprises a plurality of solid supports.

A14. The device of any one of the preceding embodiments, further comprising at least one additional reagent disposed in the inner cavity.

A15. The device of any one of the preceding embodiments, wherein the at least one additional reagent measures an analyte in the biological sample.

B1. A method of reducing interfering compounds in a biological sample comprising: (a) obtaining a biological sample directly or indirectly from a subject; (b) adding the biological sample to a device to reduce interfering compounds in a biological sample comprising: a receptacle having inner walls defining an inner cavity to contain a biological sample; and a binding agent attached to a solid support, the binding agent capable of binding a target within the biological sample; wherein binding of the target to the binding agent reduces the amount of target in the biological sample; and (c) incubating the biological sample in the device to permit the binding agent to bind the target; wherein the amount of target in the biological sample is reduced.

B2. The method of embodiment B1, wherein the binding agent is attached to the solid support via a linker.

B3. The method of any one of embodiments B1-B2, wherein the binding agent binds the target with a dissociation constant (K_{d}) of about 10⁻¹⁰ M or less.

B4. The method of any one of embodiments B1-B3, wherein the binding agent binds the target with a dissociation constant (K_{d}) of about 10⁻¹⁴ M or less.

B5. The method of any one of embodiments B1-B4, wherein the biological sample comprises blood, plasma, or any combination thereof.

B6. The method of any one of embodiments B1-B5, wherein the binding agent comprises streptavidin or avidin.

B7. The method of any one of embodiments B1-B6, wherein the binding agent is an antibody.

B8. The method of any one of embodiments B1-B7, wherein the target interferes with at least one analytical test to be performed on the biological sample.

B9. The method of any one of embodiments B1-B8, wherein the target comprises biotin.

B10. The method of any one of embodiments B1-B9, wherein from about 1.0 μL to about 1.0 L of biological sample are added to the device.

B11. The method of any one of embodiments B1-B10, wherein from about 1 mL to about 50 mL of biological sample are added to the device.

B12. The method of any one of embodiments B1-B11, wherein the biological sample is incubated in the device from about 1 minute to about 24 hours.

B13. The method of any one of embodiments B1-B12, wherein the biological sample is incubated in the device at a temperature from about 15° C. to about 30° C.

B14. The method of any one of embodiments B1-B13, wherein the incubating step further comprises agitating the device to coat the solid support with the biological fluid.

B15. The method of any one of embodiments B1-B4, wherein at least a portion of at least one inner wall of the inner cavity comprises the solid support.

B16. The method of any one of embodiments B1-B15, wherein the solid support is a fiber, filament, tubule, bead, mesh, capillary, cartridge, membrane, resin, matrix, or any combination thereof.

B17. The method of any one of embodiments B1-B16, further comprising separating at least a portion of the biological sample from the solid support after the incubating step (c).

B18. The method of any one of embodiments B1-B17, wherein substantially all of the bound target remains attached to the solid support upon separating at least a portion of the biological sample from the solid support.

B19. The method of any one of embodiments B1-B18, wherein the target is reduced by at least 25% in the biological sample.

B20. The method of any one of embodiments B1-B19, wherein the target is reduced by at least 50% in the biological sample.

B21. The method of any one of embodiments B1-B20, wherein the target is reduced by at least 75% in the biological sample.

B22. The method of any one of embodiments B1-B21, wherein the target is reduced by at least 95% in the biological sample.

C1. A kit comprising: a device to reduce interfering compounds in a biological sample comprising: a receptacle having inner walls defining an inner cavity to contain a biological sample; and a binding agent attached a solid support, the binding agent capable of binding a target within the biological sample; wherein binding of the target to the binding agent reduces the amount of target in the biological sample; and instructions for use.

C2. The kit of embodiment C1, wherein the binding agent is attached to the solid support via a linker.

C3. The kit of any one of embodiments C1-C2, wherein the binding agent binds the target with a dissociation constant (K_{d}) of about 10⁻¹⁰ M or less.

C4. The kit of any one of embodiments C1-C3, wherein the binding agent binds the target with a dissociation constant (K_{d}) of about 10⁻¹⁴ M or less.

C5. The kit of any one of embodiments C1-C4, wherein the biological sample comprises blood, plasma, or any combination thereof.

C6. The kit of any one of embodiments C1-05, wherein the binding agent comprises streptavidin or avidin.

C7. The kit of any one of embodiments C1-C6, wherein the binding agent is an antibody.

C8. The kit of any one of embodiments C1-C7, wherein the target interferes with at least one analytical test to be performed on the biological sample.

C9. The kit of any one of embodiments C1-C8, wherein the target comprises biotin.

### EXAMPLES

### Example 1

In typical competitive immunoassays for small molecules such as free thyroxine (fT4), free triiodothyronine (fT3), testosterone, estradiol, and cortisol, biotin interference can block the assay signal. Because the signal is inversely proportional to the analyte concentration in competitive assays, biotin can cause falsely high results.

In the two-site “sandwich” immunoassay format typically used for larger protein analytes, such as thyroid stimulating hormone (TSH), thyroglobulin, follicle-stimulating hormone (FSH), lutenizing hormone (LH), insulin, and autoantibodies, excess biotin competes with the biotinylated complex, causing a reduction in signal and a falsely lower result. For example, these two types of interference can lead to a false detection of Graves thyrotoxicosis, characterized by increased fT4 and suppressed TSH.

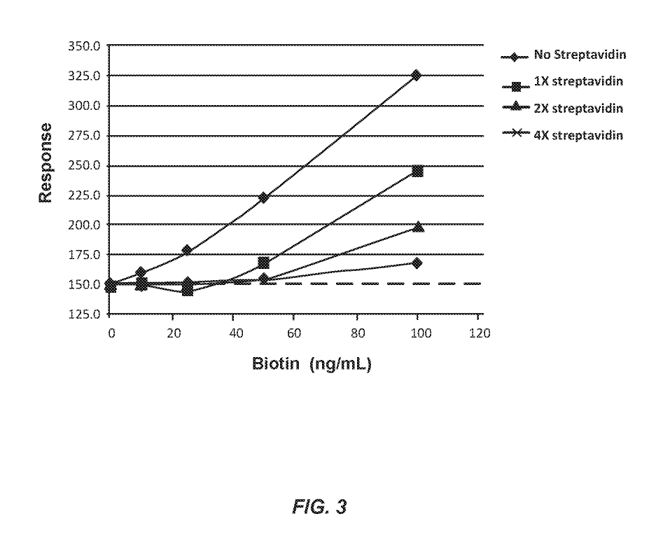

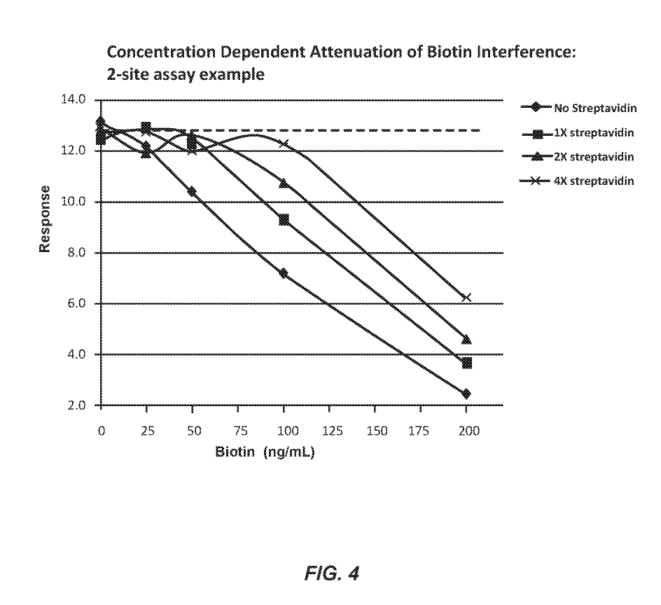

An experiment was performed using a competitive assay format, showing inhibition of positive interference by biotin for the measurement of free triiodothyronine (fT3) (Table 1 and FIG. 3). A second experiment was performed using a sandwich format showing a reduction of negative interference by biotin for the measurement of Thyroid Stimulating Hormone (TSH) (Table 2 and FIG. 4) using a two site “sandwich” assay design.

For both sets of experiments, pooled human samples were spiked with biotin at various concentrations and tested. Biotin interference in the absence of streptavidin occurs in a dose dependent fashion (FIGS. 3 and 4). The black dotted line for the sandwich assay (FIG. 4) indicates theoretical response level in the absence of biotin interference.

Streptavidin was conjugated onto the solid surface of microtiter plate wells. Unbound streptavidin was washed off and bound streptavidin stabilized. Residual unbound streptavidin was removed by washing; it is important to remove residual unbound streptavidin as it will interfere with the assay. Biotin-spiked samples were incubated in streptavidin wells to extract biotin from the samples. Sample volumes in the wells were titrated; with decreasing volume such that the streptavidin-to-biotin ratio is increased.

In the sandwich assay format capture antibody and signal antibody were used to bind to TSH antigen. Only the sandwich of capture and signal will give rise to antigen. Biotin can interfere with this complex formation. As shown in Table 2 and FIG. 4, increasing the amount of streptavidin in the samples reduced the level of interference by biotin.

A similar assay may be performed using anti-biotin in place of streptavidin, again washing away any excess anti-biotin antibody.

TABLE 1
T3 Samples - Competitive Displacement Assay

| | | | Plus Streptavidin | | Plus Streptavidin | | Plus Streptavidin | |
|---|---|---|---|---|---|---|---|---|
| | | | (120 μL | | (60 μL | | (120 μL | |
| | No Streptavidin | | sample/well) | | sample/well) | | sample/well) | |
| | No Streptavidin | | 1X Streptavidin | | 2X Streptavidin | | 4X Streptavidin | |
| Biotin | Mean | | Mean | % | Mean | % | Mean | % |
| (ng/mL) | (ng/dL) | % recovery | (ng/dL) | recovery | (ng/dL) | recovery | (ng/dL) | recovery |
| 0 | 151.1 | 100 | 148.3 | 98 | 151.8 | 100 | 151.0 | 100 |
| 10 | 160.4 | 106 | 151.5 | 100 | 150.1 | 99 | 149.6 | 99 |
| 25 | 178.4 | 118 | 145.8 | 97 | 150.1 | 99 | 151.8 | 100 |
| 50 | 222.7 | 147 | 168.3 | 111 | 154.7 | 102 | 155.6 | 103 |
| 100 | 325.2 | 215 | 245.2 | 162 | 198.5 | 131 | 168.3 | 111 |

TABLE 2
TSH Samples - Sandwich Assay

| | | | Plus Streptavidin | | Plus Streptavidin | | Plus Streptavidin | |
|---|---|---|---|---|---|---|---|---|
| | | | (120 μL | | (60 μL | | (120 μL | |
| | No Streptavidin | | sample/well) | | sample/well) | | sample/well) | |
| | No Streptavidin | | 1X Streptavidin | | 2X Streptavidin | | 4X Streptavidin | |
| Biotin | Mean | | Mean | % | Mean | % | Mean | % |
| (ng/mL) | (ng/dL) | % recovery | (ng/dL) | recovery | (ng/dL) | recovery | (ng/dL) | recovery |
| 0 | 13.2 | 100 | 12.5 | 95 | 12.8 | 97 | 13.0 | 99 |
| 10 | 12.2 | 92 | 12.9 | 98 | 12.0 | 91 | 12.8 | 97 |
| 25 | 10.5 | 79 | 12.4 | 94 | 12.6 | 96 | 12.1 | 92 |
| 50 | 7.2 | 55 | 9.4 | 71 | 10.8 | 82 | 12.3 | 94 |
| 100 | 2.5 | 19 | 3.7 | 28 | 4.7 | 36 | 6.3 | 48 |

While the preferred embodiments of the invention have been illustrated and described, it will be appreciated that various changes can be made therein without departing from the spirit and scope of the invention. All printed patents and publications referred to in this application are hereby incorporated herein in their entirety by this reference.

1. A method of reducing the amount of a target interfering compound in a biological sample comprising:
adding a binding agent to the sample, wherein the binding agent binds the target interfering compound with a higher affinity than the binding agent binds to other components in the sample;
incubating the sample to allow a complex to form between the binding agent and the target interfering compound; and
removing the complexed target interfering compound that is bound to the binding agent.

2. The method of claim 1, wherein the binding agent is attached to a solid support.

3. The method of claim 2, wherein the solid support comprises an inner surface of a receptacle or the outer surface of a mobile solid support such as a bead.

4. The method of claim 2, wherein the binding agent is attached to the solid support via a linker.

5. The method of claim 1, wherein the binding agent binds the target interfering compound with a dissociation constant (K_{d}) of 10⁻¹⁰ M or less.

6. The method of claim 1, wherein the binding agent binds the target interfering compound with a dissociation constant (K_{d}) of 10⁻¹⁴ M or less.

7. The method of claim 1, wherein the biological sample comprises blood, plasma, or any combination thereof.

8. The method of claim 1, wherein the binding agent comprises streptavidin or avidin.

9. The method of claim 1, wherein the binding agent is an antibody.

10. The method of claim 1, wherein the target interfering compound interferes with at least one analytical test to be performed on the biological sample.

11. The method of claim 1, wherein the target interfering compound comprises biotin.

12-15. (canceled)

16. The method of claim 2, wherein the solid support is a fiber, a filament, a tubule, a bead, a mesh, a capillary, a cartridge, a membrane, a resin, a matrix, or any combination thereof.

17. The method of claim 2, further comprising separating at least a portion of the biological sample from the solid support after the incubating step (c).

18. The method of claim 17, wherein substantially all of the target interfering compound remains attached to the solid support upon separating at least a portion of the biological sample from the solid support.

19. The method of claim 1, wherein the target interfering compound is reduced by at least 25% in the biological sample.

20-22. (canceled)

23. A device to reduce a target interfering compound in a biological sample comprising a binding agent attached to a solid support, the binding agent capable of binding a target interfering compound within the biological sample, wherein binding of the target interfering compound to the binding agent reduces the amount of target interfering compound in the biological sample.

24. The device of claim 23, where the device is a receptacle having inner walls to contain the sample, and the binding agent is attached to at least one inner wall.

25. The device of claim 23, wherein the binding agent is attached to the solid support via a linker.

26. The device of claim 23, wherein the binding agent binds the target interfering compound with a dissociation constant (K_{d}) of 10⁻¹⁰ M or less.

27. The device of claim 23, wherein the binding agent binds the target interfering compound with a dissociation constant (K_{d}) of 10⁻¹⁴ M or less.

28. The device of claim 23, wherein the biological sample comprises blood, plasma, or any combination thereof.

29. The device of claim 23, wherein the binding agent comprises streptavidin or avidin.

30. The device of claim 23, wherein the binding agent is an antibody.

31. The device of claim 23, wherein the target interferes with at least one analytical test to be performed on the biological sample.

32. The device of claim 23, wherein the target interfering compound comprises biotin.

33. The device of claim 24, wherein the receptacle comprises a tube, a bag, a syringe, a catheter, a flask, a bottle, a vial, a capillary tube, a pipette, a pipette tip, a plumbing tube, a needle, a microtiter plate, a multi-well collection device, or any combination thereof.

34. The device of claim 24, wherein at least a portion of at least one inner wall of the inner cavity comprises the solid support.

35. The device of claim 23, wherein the solid support is a fiber, a filament, a tubule, a bead, a mesh, a capillary, a cartridge, a membrane, a resin, a matrix, or any combination thereof.

36. The device of claim 23, wherein the device comprises a plurality of solid supports.

37. A kit comprising:
a device to reduce target interfering compounds in a biological sample comprising a binding agent attached to a solid support, the binding agent capable of binding a target within the biological sample, wherein binding of the target interfering compound to the binding agent reduces the amount of target in the biological sample; and
instructions for use.

38-47. (canceled)
